(19) United States
(12) Patent Application Publication
(10) Pub. No.: US 2006/0293743 A1
(43) Pub. Date: Dec. 28, 2006

(54) Stent assembly

(75) Inventors: Erik Andersen, Roskilde (DK); Ning Wen, Chantilly (FR)

Correspondence Address: BIRCH STEWART KOLASCH & BIRCH, PO BOX 747, FALLS CHURCH, VA, 22040-0747, US

(73) Assignee: Cube Medical A/S, Roskilde (DK)

(21) Appl. No.: 10/531,327

(22) PCT Filed: Oct. 14, 2003
(86) PCT No.: PCT/DK03/00698

§ 371 Date: Jul. 28, 2006

(30) Foreign Application Priority Data
Oct. 14, 2002 (GB) 02238707.7

(51) Int. Cl. A61F 2/06

(52) U.S. Cl. 623/1.13; 623/901

(57) ABSTRACT

A stent assembly comprises an expandable tubular stent (21), an external surface of which is provided with a fabric (5) which may constitute a reservoir to hold drugs to be released in the intravascular system of a living. The fabric (5) may be made from a filamentary material, such as multifilament yarn (6). Electrospinning of nanofibers of, e.g., polymer may be employed for manufacturing the fabric. The stent may comprise an assembly of radially expandable, tubular elements (1) aligned along a common longitudinal axis and successively joined together in pairs by respective sets of linking members (4), e.g. forming a zigzag corrugation. The stent may be crimped onto a balloon.

### TECHNICAL FIELD

This invention relates to a stent assembly including an expandable tubular stent for Implantation in the lumen of a body duct in order to ensure a passage therein.

### BACKGROUND OF THE INVENTION

Such stents are used mainly in the treatment of blood vessels exhibiting stenoses, and more generally in the treatment of diseases of various anatomical ducts of the human or animal body, such as, for example, the urinary ducts, especially the urethra, or the digestive ducts, especially the oesophagus.

The percutaneous implantation of an expandable tubular stent in a stenotic blood vessel is generally recommended, for example after a conventional angioplasty procedure, for preventing the dilated vessel from closing up again spontaneously or for preventing its occlusion by the formation of a new atheromatous plaque and the possible recurrence of stenosis.

There are many different arrangements of tubular stent, and the present invention is not limited to any specific type; however, for the purpose of explanation, the invention is described in relation to a particular known type of expandable tubular stent which consists of an assembly of radially expandable, tubular elements aligned along a common longitudinal axis and successively joined together in pairs by respective sets of linking members. Such a stent is disclosed, for example, in international patent application WO 98/58600 in which each of the tubular elements consists of a strip forming a zigzag corrugation defining bent extreme portions which are successively connected together in pairs in opposite directions by rectilinear intermediate portions. By virtue of this zigzag corrugation, the stent is expandable between a first, unexpanded state, enabling it to be implanted percutaneously by means of an insertion device of reduced diameter, and a second, expanded state, in which the stent makes it possible to ensure a passage in the lumen of the body duct. Stents of this type are also disclosed in international patent applications WO 96/26689 and WO 98/20810.

To install the stent, it is placed in the unexpanded state on an angioplasty balloon catheter. Once in place, the balloon is inflated in order to cause the stent to expand. Alternatively, the stent may be made from a material which has a recovery capacity, so that the stent may automatically expand, once in place.

### SUMMARY OF THE INVENTION

According to the present invention, there is provided a stent assembly comprising an expandable tubular stent, on the cylindrical external surface of which is a fabric. The fabric may cover the whole of the cylindrical external surface, or just a part.

As will be explained in more detail, the fabric can be used as a reservoir to hold drugs, in particular those intended for sustained release in the period after deployment of the stent. The fabric can also be used to improve the support provided by the stent on the wall of the vessel or duct being treated.

The fabric is made from filamentary material such as fibre and its construction may be woven, non-woven or knitted. The filamentary material may be of a continuous nature, such as would be used for manufacturing a woven or knitted fabric, or in short random lengths such as would be used in non-woven mat products, or paper-based products, such as tissue.

The fabric is initially applied to the stent when in its unexpanded condition and must therefore be itself capable of expansion with the stent. This can be achieved either by making the fabric itself expandable, or, where the fabric is not itself sufficiently expandable, to provide folds in the fabric covering the unexpanded stent so that, as the stent dilates, the folds will open to the intended full size of the stent. A combination of these techniques can be used.

The fabric can be made expandable either by virtue of its inherent construction—certain knitted fabrics, in particular, show high expansion capabilities—or by using elastic filamentary material in its construction, or both. Examples of suitable filamentary material include polymers such as polyurethane, polyamide, gelatine, silicone or agar. In order to provide the appropriate characteristics of elasticity, the filamentary material should have a high capability of elongation and may be pre-stretched.

The openness of the fabric construction may be varied to suit the circumstances. Close construction fabrics can provide better (more homogeneous) support for the vessel wall and in fact the construction can be sufficiently close to provide reservoirs for liquid-based drugs between adjacent strands of the filamentary material making up the fabric. Close construction fabrics, however, can lead to problems where the vessel being treated has side branches located within the treatment site. In such a case a more open construction of fabric is preferred in order to avoid blockage of the side branch due to the fabric. Such open fabrics can still provide a reservoir for liquid-based drugs, by making the strands of filamentary material of multi-filament type. Such filamentary material can act as a wick to draw into itself by capillary action a liquid-based drug, and subsequently act as a reservoir for sustained release of the drug.

There are various possible ways of applying the fabric to the stent. The fabric may be applied to the stent as a finished fabric, for example in the form of a sheet-like fabric which is wrapped around the stent, or in the form of a length of tubular fabric in which the stent is placed. Alternatively, the fabric may be manufactured in situ on the stent by winding one or more strands of filamentary material over the stent in a pattern suitable to create the desired fabric.

In one embodiment, the fabric is applied by spinning of nanofibers, preferably electrospinning of such nanofibers, which consolidate to form the fabric. It has been found that such spinning of nanofibers may relatively easily or accurately controlled. It has also been found that fabrics produced by electrospinning of nanofibers have a low surface friction, and that such fabrics are well-suited as reservoirs to drugs, i.e. medical tubings in which the electrospun portions thereof constitute reservoirs for holding drugs. Various polymer-based materials may form the nanofibers, including polymer solutions and polymer melts. Applicable polymers are: nylon, fluoropolymers, polyolefins, polyimides, and polyesters. Further, carbon may be used as a fiber-forming material. The art of electrospinning of nanofibers has developed considerably In recent years. U.S. Pat. No. 6,382,526 discloses a process and apparatus for the production of nanofibers, which process and apparatus are useful in electrospinning the present fabric, and U.S. Pat. No. 6,520,425 discloses a nozzle for forming nanofibers. It should be understood that the processes and apparatuses of the aforementioned US patents may be applicable in the method according to the present invention, but that the scope of protection is not restricted to those processes and apparatuses. Typically, the diameter of the nanofibers is in the range of 2 to 4000 nanometers, preferably 2 to 3000 nanometers.

The properties of the fabric produced by nanospinning the present invention may be determined by the fiber-forming materials and/or by production parameters, such as voltage of electrodes in the electrospinning process, distance between high-voltage and low-voltage electrodes, rotational speed of the tubing (or of a core wire around which the tubing is manufactured), electrical field intensity, corona discharge initiation voltage or corona discharge current.

### BRIEF DESCRIPTION OF THE DRAWINGS

In order that the invention may be better understood, several embodiments of the Invention will now be described by way of example only and with reference to the accompanying drawings in which:

FIG. 1 is a side view of a first embodiment of a stent assembly according to the invention;

FIG. 2 is a perspective view of the stent assembly of FIG. 1;

FIG. 3 is a side view of the stent of FIG. 1, in its expanded condition;

FIG. 4 is a view similar to FIG. 3, showing a second embodiment of the stent assembly of the present invention;

FIG. 5 is a perspective view of the expanded stent assembly of FIG. 4;

FIG. 6 is a diagrammatic side view of the stent assembly of the present invention deployed in an artery having side branches;

FIGS. 7 and 8 are perspective views illustrating two methods of applying the fabric to the stent;

FIG. 9 is a side view illustrating the method of applying the fabric simultaneously to two stents; and

FIG. 10 is an enlarged view of part of FIG. 4.

### DETAILED DESCRIPTION OF THE DRAWINGS

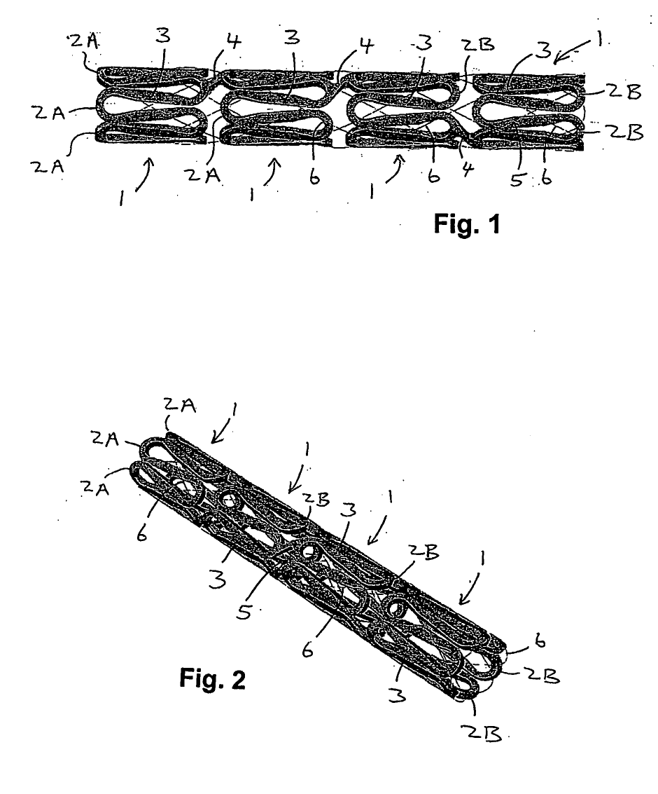

FIGS. 1 and 2 show a first embodiment of a stent assembly according to the invention in side and perspective views respectively. The assembly comprises a stent consisting of an elongate, approximately tubular body or frame defined by a plurality of tubular elements 1 aligned along a common longitudinal axis and successively joined together by a plurality of linking members 4. A stent of four elements is shown in FIGS. 1 and 2, however typical stents can have as few as 3 elements, or as many as 20 elements, or even more, depending upon the circumstances.

Each tubular element 1 consists of a strip forming a zigzag corrugation defined by bent portions 2 which are successively connected together in pairs In opposite directions by rectilinear intermediate portions 3. For each tubular element, there can thus be defined two sets of bent portions: bent portions 2A connecting the rectilinear portions 3 on one end of the tubular element; and bent portions 2B connecting the rectilinear portions 3 on the opposite end of the tubular element. In FIG. 1 the bent portions 2A and 2B are arbitrarily shown on the left and right hand ends respectively of each tubular element.

Advantageously, for a given tubular element, the rectilinear portions 3 are all of the same length and the bent portions are all identical and approximately semi-circular. Thus the corrugation advantageously has a uniform shape. The tubular elements 1 are joined in such a way that the corrugations of adjacent tubular elements 1 are in phase.

Except at the ends, each tubular element 1 is joined to its neighbour by a single linking member 4. The number of linking members, however, need not be just one, and can range from none at all, to several, the exact number depending upon the structure of the tubular elements—in particular the number of corrugations—as well as the particular characteristics required of the stent (where there are no linking members, the tubular elements are, of course, independent of one another).

It will be understood that the above-described design of stent is given by way of example only; the stent assembly of the present invention can use any one of a large number of different designs of expandable tubular stent. The assembly further comprises a layer 5 of fabric material which completely covers the cylindrical external surface of the stent. The particular fabric material shown is of open construction and is made from multi filament yarn 6, for example polymer yarn. Optionally a liquid-based drug is soaked into the yarn and held therein by virtue of its multi-filamentary construction. Examples of suitable fabrics include non-woven fabrics such as tissue, woven fabrics and knitted fabrics. The drug used will depend upon the particular requirements. For example drugs capable of discouraging restenosis could be used.

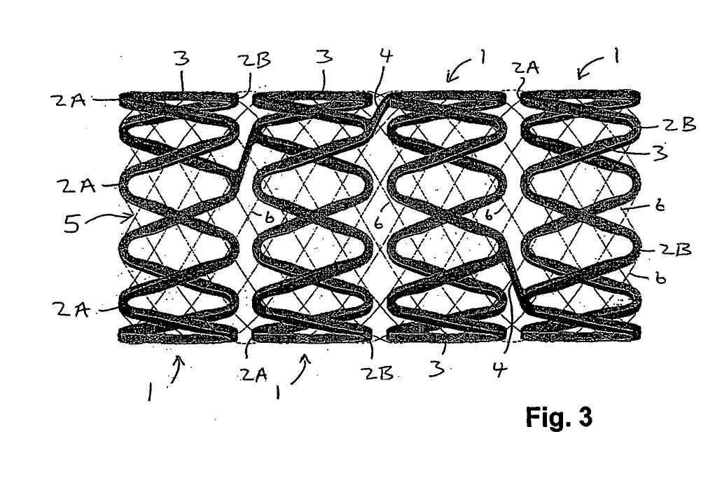

The yarn 6 is elastic and in particular has the property of high elongation. It may be pre-stretched. The yarn 6 needs to be elastic because, as the stent expands during deployment, the yarn 6 is stretched, and must be capable of doing this without breaking, or significantly hindering the expansion of the stent. FIG. 3 illustrates the expanded condition of the stent.

When fully expanded the stent supports the vessel wall and prevents it collapsing. The fabric layer 5 becomes trapped between the stent and the vessel wall and helps to support the vessel wall. In addition, it will be seen that any drugs loaded into the fabric will be applied directly to the inner wall of the vessel, as well as being available to pass into the liquid following along the vessel due to the open nature of the stent.

Even without the assistance of the fabric layer 5, the particular design of stent described above is able to provide a reasonably homogeneous support to the wall of the vessel being treated because the corrugations remain approximately in phase with one another during dilation. However, it is clear from FIG. 3 that the use of an external fabric layer 5 enables still better support to be provided for the vessel wall because the fabric passes across the gaps between the tubular elements 1 and thus provides a degree of support even between the elements 1, where the vessel wall would otherwise be unsupported.

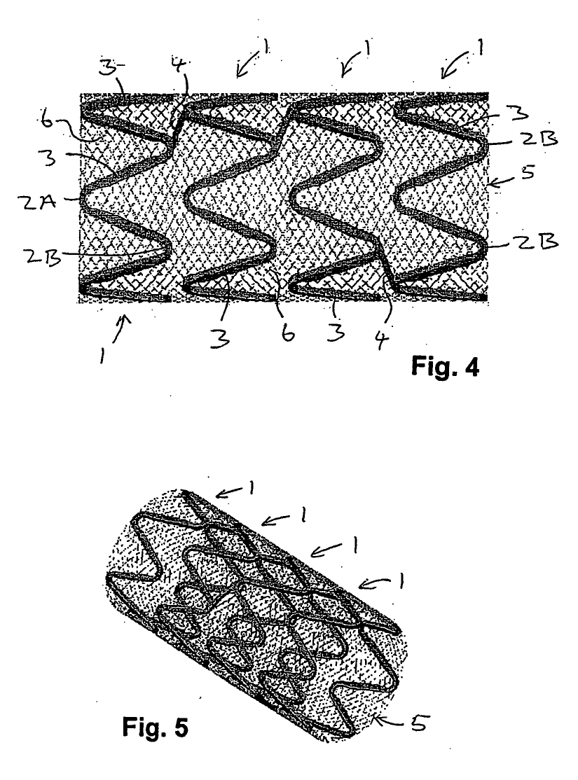

FIGS. 4 and 5 illustrate, in its expanded condition, an alternative embodiment utilising the same stent as that illustrated in FIGS. 1 to 3, but with a layer 5 of a fabric having a relatively fine mesh—i.e. a more closed construction. Such a fabric is capable of providing still greater support for the vessel wall between the components of the tubular elements 1.

FIG. 10 illustrates part of FIG. 4, on an enlarged scale, to assist the illustration of typical dimensions. In FIG. 10, the yarn diameter A is preferably in the range from 0.005 mm to 0.05 mm in diameter, typically 0.01 mm in diameter, in the unexpanded condition of the stent. During expansion of the stent, the yarn stretches to a diameter preferably in the range 0.001 mm to 0.01 mm, typically 0.005 mm.

The yarn separation distance B is also illustrated in FIG. 10. Typically this ranges from zero (i.e. yarns touching) to 0.05 mm in the unexpanded condition of the stent. After stent dilation the distance B increases typically to a range of from 0.1 mm to 0.4 mm with a mean value typically of about 0.2 mm.

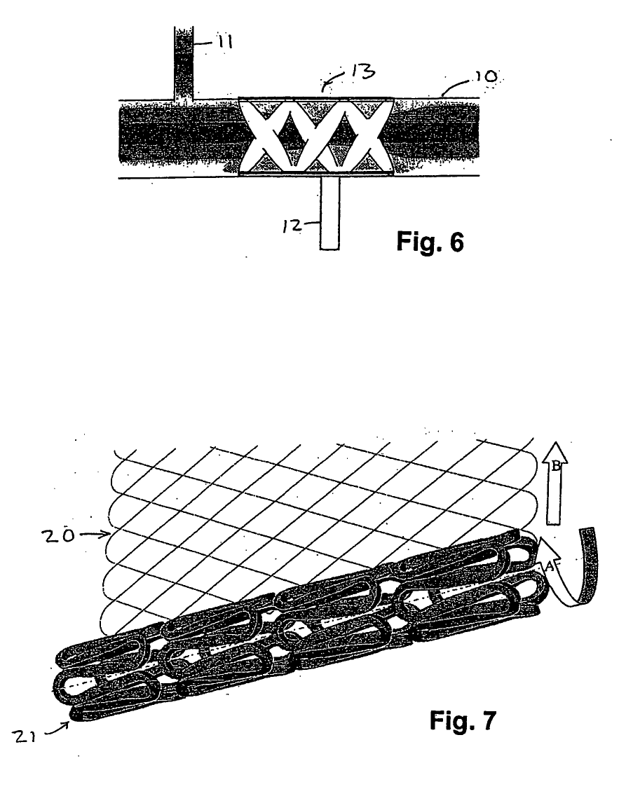

It should be noted that fine mesh fabrics cannot be used in cases where the vessel has side branches within the area of the treatment site. FIG. 6 illustrates the problem diagrammatically. In FIG. 6 there is shown an artery 10 to be treated, said artery having side branches 11 and 12. A stent assembly 13 according to the invention is illustrated in diagrammatic outline extending across the side branch 12. Once the stent assembly is fully deployed, any blood flowing into or out of the side branch 12 has to pass through the fabric layer 5 and there is therefore a risk that the side branch 12 may block. To prevent this the fabric should have a reasonably open construction.

FIGS. 7 to 9 illustrate various aspects of the manufacture of a stent assembly according to the invention.

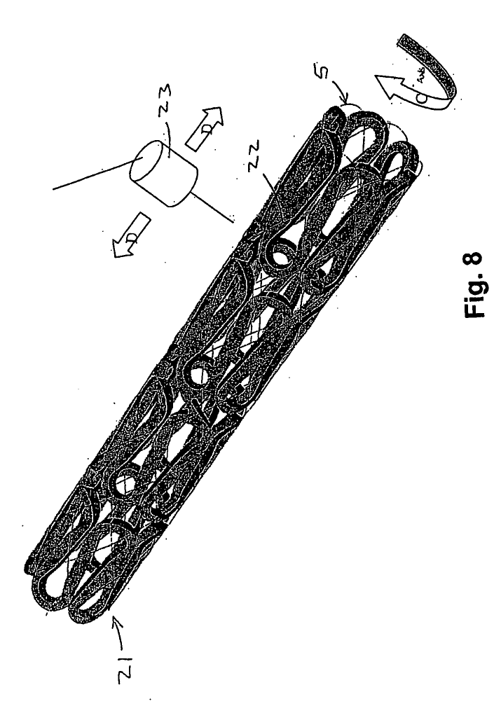

The stent itself can be manufactured by any of the conventional methods (see below). Following this, the fabric layer can be applied to the unexpanded stent by various methods, two of which are illustrated in FIGS. 7 and 8 respectively. In FIG. 7, a pre-formed fabric in the form of a sheet 20 is rolled or wound over the outer surface of a tubular stent 21. The resultant layer covering the stent can be formed of one or more turns of the fabric, according to the requirements; obviously the greater the number of turns the closer the construction of the ultimate fabric layer.

In FIG. 8 a single multi-filament yarn 22 is applied to the stent 21 and built up to form a fabric layer. Item 23 may be a guide for yarn taken from a distant reel (not shown). As the yarn is payed out, the guide reciprocates backwards and forwards along an axis parallel to, but spaced from, the longitudinal axis of the stent. At the same time, the stent rotates about its longitudinal axis so that the yarn is laid down onto the exterior surface of the stent in a series of helical sections which cumulatively make up the fabric. In an alternative, the item 23 takes the form of a pen extruder which directly extrudes the yarn onto the stent surface.

The technique used in FIG. 8 may be modified to thread the yarn through the components of the stent so that the fabric layer 5 is securely anchored to the stent. In this case, not all of the yarn will cover the external surface of the stent, but some will pass over parts of the internal cylindrical surface of the stent in order to form loops around the stent components.

In another method of manufacture, the fabric may be pre-formed into the shape of a tube into which the unexpanded stent is placed. The size of the fabric tube may be slightly smaller than the stent over which it is fitted, so that, even in the unexpanded state of the stent, the fabric layer exerts a slight inward gripping action. Alternatively, the fabric tube may have a diameter more nearly matching that of the expected expanded diameter of the stent, and be longitudinally folded to a smaller diameter when fitted over the unexpanded stent. Thus, when the stent expands, the fabric tube will unfold so that, when the stent is fully expanded, the fabric tube is fully unfolded.

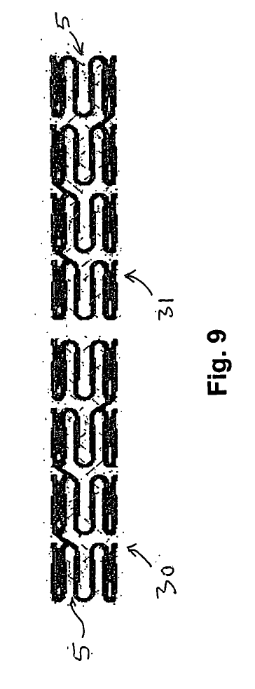

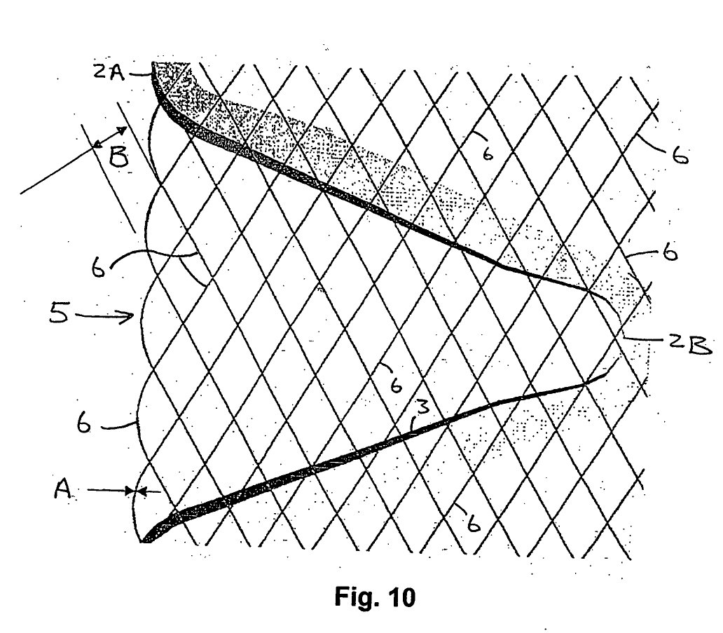

It would also be possible to use any of the techniques described above to apply the fabric layer to multiple stents simultaneously, in order to optimise production. As an example, FIG. 9 illustrates two axially-aligned stents 30,31 which have been covered simultaneously. After covering, the individual stents are separated by cutting through the fabric at the appropriate point or points—FIG. 9 shows the stents after separation.

It is stated above that the fabric is applied to the stent when in its unexpanded condition. By this is meant prior to its expansion at the treatment site during deployment. If the stent is to be expanded by means of a balloon, then it is likely that the stent will be crimped onto the balloon in order to secure the stent in place on the balloon during its, often tortuous, passage to the treatment site. The crimping process involves compressing the stent onto the balloon, which results in a reduction in its radial diameter from its “as-cut” condition—i.e. the condition in which it leaves the manufacturing process (see below). The fabric layer can be applied when the stent is either in the “as-cut” condition, or in the crimped condition since both these can be regarded as unexpanded conditions.

During deployment, the stent assembly is expandable between an unexpanded state (in practice, probably the crimped condition mentioned above), in which it is able to be guided inside the lumen through a body duct, such as a blood vessel, for example, and an expanded state, in which the fabric layer 5 covering the stent, after a uniform expansion, comes into contact with the inner wall of the body duct, defining a passage of approximately constant diameter inside said duct. In the expanded state, the fabric layer 5 is thus trapped between the outer surface of the stent and the inner wall of the body duct.

The stent will generally be forcibly expanded mechanically under the action of a force exerted radially outwards, for example under the effect of the inflation of a balloon. However, the stent may be of the “auto-expandable” type, I.e. capable of changing by itself from a first, unexpanded condition under stress, enabling it to be guided through the body duct, to a second, expanded, working condition.

The stent may be made of any material compatible with the body duct and the body fluids with which it may come into contact.

In the case of an auto-expandable stent, it will be preferable to use a material with a recovery capacity, for example, stainless steel, Phynox® or nitinol.

In the case of a stent utilising a forced expansion, a material with a low elastic recovery capacity may be used to advantage. Examples are metallic materials such as tungsten, platinum, tantalum, gold, or stainless steel.

The stent may be manufactured from a hollow tube with an approximately constant thickness corresponding to the desired thickness. The pattern of tubular elements and linking members may be formed either by laser cutting followed by electrochemical polishing, or by chemical or electrochemical treatment.

The stent may alternatively be manufactured from a sheet of approximately constant thickness corresponding to the desired thickness of the stent. The geometric configuration of the stent can be obtained either by laser cutting followed by electrochemical polishing, or by chemical or electrochemical treatmenmt. The sheet cut in this way is then rolled up to form a cylinder and welded to give the desired final structure.

The stent assembly which has been described can be inserted in a manner known per se. In the case of a stent utilising mechanically forced expansion, the insertion system will preferably comprise a balloon-tip catheter onto which the stent will be crimped in the unexpanded state before being introduced into an insertion tube for guiding it to the site to be treated.

It should be noted that the stent assembly described herein can be used also for the fixing of implants, particularly casings made of woven, non-woven or expanded porous polymers, and is particularly useful in the isolation of aneurisms, where the fibre layer can provide an effective reinforcing mesh over the entrance to the aneurism. Any drugs which are soaked into the fabric may be such as to assist in the process of isolating the aneurism.

1. A stent assembly comprising a tubular stent, an external surface of which is provided with a fabric, characterized in that the fabric has been pre-formed into the shape of a tube, into which the stent has been placed in the unexpanded state of the stent, the fabric tube having an inner diameter which is smaller than the outer diameter of the stent when the tube is placed on the stent to achieve an inward gripping action of the fabric tube on the unexpanded stent.

2. A stent assembly according to claim 1, wherein the fabric tube has been manufactured with an inner diameter which is smaller than the outer diameter of the unexpanded stent.

3. A stent assembly according to claim 1, wherien the fabric tube has been longitudinally folded to a smaller diameter than the outer diameter of the stent when fitted over the unexpanded stent.

4. A stent assembly according claim 1, wherein the fabric constitutes a reservoir to hold drugs.

5. A stent assembly according to claim 1, wherein the fabric is made from a filamentary material.

6. A stent assembly according to claim 5, wherein the filamentary material includes at least one polymer.

7. A stent assembly according to claim 6, wherein the at least one polymer is selected from the group consisting of: polyurethane, polyamide, gelatine, silicone and agar.

8. A stent according to claim 5 wherein the fabric is made from a multifilament yarn.

9. A stent assembly according to claim 5, wherein at least a portion of the fabric is produced by spinning of nanofibers.

10. A stent assembly according to claim 9, wherein said portion is produced by electrospinning.

11. A stent assembly according to claim 9, wherein the diameter of the nanofibers is in the range of 2 to 4000 nanometers, such as in the range of 2 to 3000 nanometers.

12. A stent assembly according to claim 9, wherein the nanofibers are made from a polymer.

13. A stent assembly according to claim 12, wherein the nanofibers are made from a material selected from the group consisting of: nylon, fluoropolymers, polyolefins, polyimides, and polyesters.

14. A stent assembly according to claim 1, wherein the fabric has an openness which allows the fabric to serve as a reservoir for liquid-based drugs.

15. A stent assembly according to claim 1, wherein the tubular stent comprises an assembly of radially expandable, tubular elements aligned along a common longitudinal axis and successively joined together in pairs by respective sets of linking members.

16. A stent assembly according to claim 15, wherein each tubular element exists essentially of a strip forming a zigzag corrugation.

17. A stent assembly according to claim 1, wherein the fabric completely covers the cylindrical external surface of the stent.

18. A stent assembly according to claim 1, wherein the stent is crimped onto a balloon for expanding the stent.

19. A stent assembly according to claim 1, wherein the stent is auto-expandable.

20. A stent assembly according to claim 19, wherein the stent is made essentially from a material selected from the group consisting of: stainless steel, Phynox®, and nitinol.

21. A stent assembly according to claim 1, wherein the stent is expandable by forced expansion, the stent being made essentially from a metallic material.

22. A stent assembly according to claim 21, wherein the metallic material is selected from the group consisting of: tungsten, platinum, tantalum, gold, and stainless steel.

23. A method of manufacturing a stent assembly according to claim 1, comprising the steps of:
manufacturing the stent;
applying the fabric to the stent, characterized in that the fabric is preformed into the shape of a tube before the fabric is applied to the stent, the step of applying the fabric to the stent comprising placing the unexpanded stent into the fabric tube, the fabric tube having an inner diameter which is smaller than the outer diameter of the stent when the tube is placed on the stent to achieve an inward gripping action of the fabric tube on the unexpanded stent.

24. A method according to claim 23, wherein the fabric tube is preformed with an inner diameter which is smaller than the outer diameter of the unexpanded stent.

25. A method according to claim 23, wherein the step of applying the fabric tube to the stent comprising longitudinally folding the fabric tube to a smaller diameter than the outer diameter of the unexpanded stent.

26. A method according to claim 23, wherein the fabric is manufactured by spinning of nanofibers.

27. A method according to claim 26, wherein the step of spinning comprises electrospinning.

28. A method according to claim 26, wherein the diameter of the nanofibers is in the range of 2 to 4000 nanometers.

29. A method according to claim 26, wherein the step of spinning comprises feeding a first fiber-forming material into a nozzle for forming nanofibers by using a pressurized gas stream, and ejecting the first fiber-forming material from an exit orifice of the nozzle in the form of a plurality of strands of said first fiber-forming material that solidify and form said nanofibers.

30. A method according to claim 26, wherein the nanofibers are made from a polymer.

31. A method according to claim 30, wherein the nanofibers are made from a material selected from the group consisting of: nylon, fluoropolymers, polyolefins, polyimides, and polyesters.

32. A method according to claim 23, wherein the stent is manufactured from a hollow tube, in which a pattern of tubular elements and linking elements is formed.

33. A method according to claim 23, wherein the step of manufacturing the stent comprises rolling up of a sheet of material to form the tube, and securing adjoining edge portions of the sheet together.

34. A method of preparing a stent assembly according to claim 1, comprising the step of providing a drug to the fabric.
